(19) United States
(12) Patent Application Publication
(10) Pub. No.: US 2007/0050002 A1
(43) Pub. Date: Mar. 1, 2007

(54) Intra-thecal catheter and method for cooling the spinal cord and brain

(76) Inventor: John Elefteriades, Guilford, CT (US)

Correspondence Address: WELSH & FLAXMAN LLC, 2000 DUKE STREET, SUITE 100, ALEXANDRIA, VA, 22314, US

(21) Appl. No.: 11/518,217

(22) Filed: Sep. 11, 2006

Related U.S. Application Data
(63) Continuation-in-part of application No. 11/052,479, filed on Feb. 8, 2005.

(51) Int. Cl. A61F 7/00

(52) U.S. Cl. 607/105

(57) ABSTRACT

A method for cooling of the brain includes the steps of positioning a cooling catheter within a ventricular cavity of the brain, the catheter including an inlet channel and outlet channel providing for the closed flow of cooling fluid into and out of the catheter, and cooling the catheter and ventricular cavity through the closed flow of cooling fluid through the catheter. An alternate method for cooling of the brain including the steps of positioning a cooling catheter within a spinal canal, the catheter including an inlet channel and outlet channel providing for the closed flow of cooling fluid into and out of the catheter, and cooling the catheter and brain through the closed flow of cooling fluid through the catheter.

### CROSS REFERENCE TO RELATED APPLICATIONS

This application is a continuation-in-part of U.S. patent application Ser. No. 11/052,479, entitled “INTRA-THECAL CATHETER AND METHOD FOR COOLING THE SPINAL CORD”, filed Feb. 8, 2005, which is currently pending.

### BACKGROUND OF THE INVENTION

1. Field of the Invention

The present invention relates to a method and apparatus for cooling the spinal cord and the brain. In particular, the invention relates to a method and apparatus for cooling of the spinal cord for descending and thoracoabdominal aortic surgery through the utilization of an intra-thecal catheter.

2. Description of the Prior Art

Despite advances in spinal cord protection, paraplegia continues to be a serious complication of descending and thoracoabdominal aortic operations. Paraplegia has been a serious and vexing problem since the advent of direct thoracic aortic surgery some 40 years ago. Paraplegia continues to devastate the lives of patients undergoing surgery for thoracic aortic aneurysm; in cases of post-operative paraplegia, mortality is high and, even in survivors, quality of life is devastated.

Spinal ischemia is a known postoperative complication following aortic surgeries. The incidence of spinal cord ischemia during aortic surgery is typically over 10%. During thoracic or thoracoabdominal aortic aneurysm repair, for example, the spinal arteries, which provide blood supply to the spinal cord, are often severed from the diseased aorta, and some but not all ate later resutured to a prosthetic graft. As a result, blood flow to the spinal cord is reduced. When aortic clamp time and consequent reduction of spinal perfusion lasts more than 45 minutes, spinal ischemia ensues, often resulting in paralysis.

In recent years, there is a general sense that improvements are being made in better preventing paraplegia. Multiple advances have expanded the anti-paraplegia armamentarium. Re-discovery of left atrial-to-femoral artery perfusion for descending and thoracoabdominal operations permits reliable perfusion of the lower body and spinal cord. Collagen-impregnated grafts have improved hemostasis and inherent handling characteristics of available prostheses. Identification and re-implantation of spinal cord arteries has improved. Spinal cord drainage, aimed at improving the perfusion gradient for the spinal cord, by minimizing external pressure on cord tissue, has been adopted almost universally. The advent of effective anti-fibrinolytic agents has decreased peri-operative blood loss and, consequently, led to improved hemodynamics. The importance of maintaining proximal hypertension during the cross-clamp time has been recognized. The fact that that nitroprusside administration is contra-indicated during surgery, because its administration can lead to increased intra-thecal pressure, has also been recognized. In addition, it has been found that by keeping blood pressure high after aortic replacement during the ICU and step-down unit stays it is possible to prevent many cases of paraplegia. It has also been found that early recognition and treatment of late post-operative paraplegia can often lead to restoration of spinal cord function; important measures include raising the blood pressure with inotropic medications and volume administration, optimization of hematocrit with blood transfusions, and re-institution of spinal cord drainage.

Yet, with all of the advances described above, and the many more advances not described herein, paraplegia has not been reduced to zero incidence. This continues to be a major issue, both clinically and medico-legally.

Cooling is known to be protective against ischemia for all body tissues, especially the brain and spinal cord. In fact, one group uses instillation of cold fluid into the intra-thecal space to produce core cooling and protect the spinal cord during aortic surgery. Cambria R P, Davison J K, Zannetti S, et al: Clinical experience with epidural cooling for spinal cord protection during thoracic and thoracoabdominal aneurysm repair, J Vasc Surg 25:234-243, 1997. Despite good local results, this technique has not been generally adopted, because of concerns about the cumbersome nature of instilling and draining fluid, and because of documented elevation in intra-thecal pressure consequent upon fluid instillation.

The experience of Kouchoukos and colleagues with the performance of descending and thoracoabdominal replacement under deep hypothermic arrest—with a near zero paraplegia rate—demonstrates vividly the powerful protective influence of hypothermia. Yet, most aortic surgeons do not utilize deep hypothermic arrest for descending and thoracoabdominal operations, out of concern for potential negative effects of deep hypothermia and prolonged perfusion in this setting.

It is also known that brain damage associated with either stroke or head trauma is worsened by hyperthermia and improved with hypothermia. As such, and as with the hypothermia treatments for the spinal canal discussed above, various researchers have attempted to utilize hypothermia in treating stroke and head trauma. However, these attempts have met with only limited success.

Of particular relevance is U.S. Pat. No. 6,699,269 to Khanna. This patent provides a method and apparatus for performing selective hypothermia to the brain and spinal cord without the need for systemic cooling. In accordance with the disclosed embodiment, a flexible catheter with a distal heat exchanger is inserted into the cerebral lateral ventricle or spinal subdural space. The catheter generally includes a heat transfer element and three lumens. Two lumens of the catheter circulate a coolant and communicate at the distal heat transfer element for transfer of heat from the cerebrospinal fluid. The third lumen of the catheter allows for drainage of the cerebral spinal fluid.

While the system disclosed in the Khanna patent generally discloses a system for spinal cord and brain cooling, Khanna offers very few details regarding the specific structures and procedures for achieving the goal of spinal cord and brain cooling. As those skilled in the art will certainly appreciate, cooling of the spinal cord or brain is not merely a matter of inserting a catheter having a heat exchanger at a distal end thereof within the space desired for cooling and hoping for the best results. Rather, detailed analysis is required so that such a system may actually function to serve the needs of patients. Khanna fails to provide the specificity required for achieving this goal. As such, Khanna may be considered in much the same category as the other prior art references as not providing a system for sufficiently addressing the goal of spinal cord and brain cooling.

As such, a need exists for a method and apparatus whereby the spinal cord and brain of an individual may be cooled with the hopes of reducing and eliminating spinal cord injuries. The present invention provides such a method and apparatus.

### SUMMARY OF THE INVENTION

It is, therefore, an object of the present invention to provide a method for cooling of the brain including the steps of positioning a cooling catheter within a ventricular cavity of the brain, the catheter including an inlet channel and outlet channel providing for the closed flow of cooling fluid into and out of the catheter, and cooling the catheter and ventricular cavity through the closed flow of cooling fluid through the catheter.

It is also an object of the present invention to provide a method for cooling of the brain including the steps of positioning a cooling catheter within a spinal canal, the catheter including an inlet channel and outlet channel providing for the closed flow of cooling fluid into and out of the catheter, and cooling the catheter and brain through the closed flow of cooling fluid through the catheter.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a cross sectional view of the catheter in accordance with the present invention.

FIGS. 2 and 3 are schematic views of alternate systems in accordance with the present invention.

FIG. 4 is a partial perspective view of the spine with a catheter in accordance with the present invention inserted therein.

FIG. 5 is a side view of the spine with a catheter in accordance with the present invention inserted therein.

FIG. 6 is a cross sectional view of spine with a catheter in accordance with the present invention inserted therein.

FIGS. 7, 8, 9 and 10 are schematics showing cooling of the brain in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limited, but merely as the basis for the claims and as a basis for teaching one skilled in the art how to make and/or use the invention.

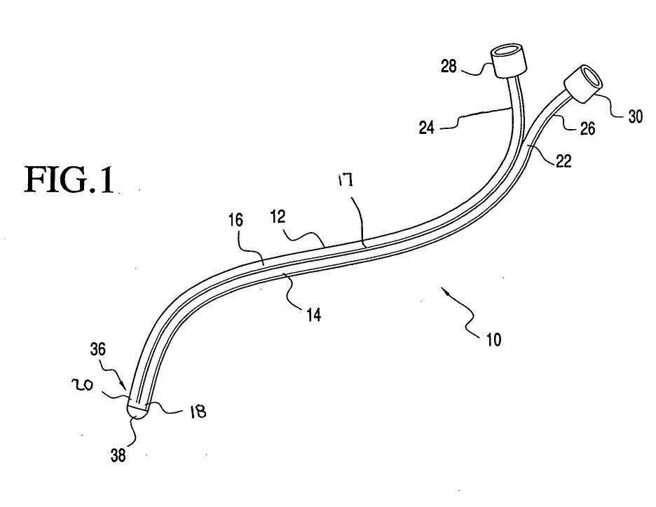

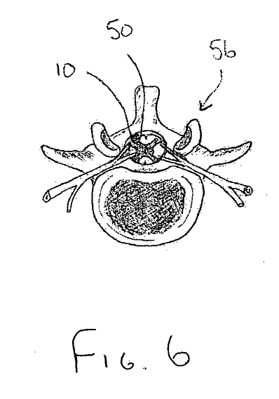

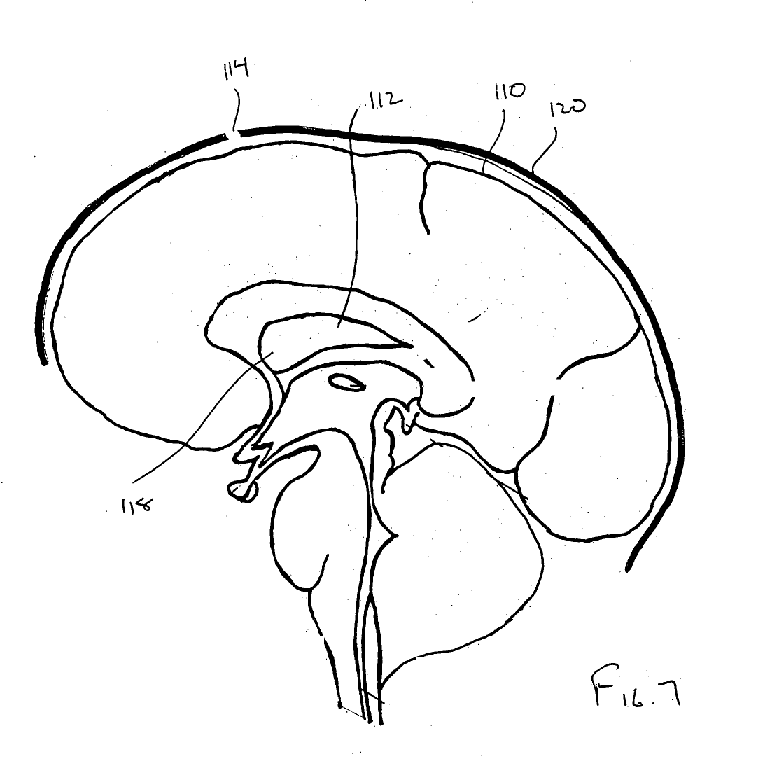

With reference to FIGS. 1 to 6, a method and apparatus for intra-thecal cooling is disclosed. The method and apparatus provide an effective mechanism for cooling the spinal cord in an effort to reduce the spinal ischemia. Generally, the present intra-thecal cooling catheter system 1 includes a closed-loop, cooling catheter 10 coupled to a cooling system 11 coupled to the catheter 10.

With regard to the intra-thecal cooling catheter 10 of the present invention, it is generally a dual lumen polyurethane catheter with a 50/50 split. That is, the catheter 10 is generally composed of a cylindrical, extruded tube 12 with two hollow semi-circular channels, that is, inlet and outlet channels 14, 16, providing for the flow of cooling fluid into and out of the catheter 10.

More particularly, and in accordance with a preferred embodiment of the present invention, the catheter 10 is approximately 3 feet long. The catheter 10 has an outer diameter of approximately 0.065 inches, an inner diameter of approximately 0.045 inches and wall thickness of approximately 0.010 inches. The septum 17 separating the inlet and outlet channels 14, 16 is approximately 0.006 inches thick.

The distal ends 18, 20 of the channels 14, 16 formed within the catheter 10 are connected so that a cooling fluid may be freely circulated within a closed loop extending through the catheter 10. In particular, cooling fluid flows down the inlet channel 14 and back up the outlet channel 16, providing cooling along the entire length of the catheter 10. At the proximal end 22 of the catheter 10, the inlet and outlet channels 14, 16 split into individual tubes. The proximal ends 24, 26 of the respective channels 14, 16 are provided with a luer connection 30, 28 for fitting tubes 32, 34 to supply (inlet) and remove (outlet) cooling fluid from the catheter 10.

The distal end 36 of the catheter 10 is sealed with an acrylic sphere 38. The acrylic sphere 38 is bonded to the distal end 36 of the catheter 10 and seals the end of the catheter 10. In accordance with a preferred embodiment of the present invention, the sphere 38 has a diameter of approximately 0.063 inches. Most importantly, it provides a smooth surface for advancing the catheter 10 through the epidural space and intra-thecal space while minimizing tissue disruption. Flow between the inlet and outlet channels 14, 16 is achieved by cutting back the septum 17 between the inlet and outlet channels 14, 16 such that fluid may freely flow between the sphere 38 and the cut back portion of the septum 17.

In accordance with a preferred embodiment of the present invention, the catheter 10 is no greater than 18 to 16 gauge and is a flexible, atraumatic cooling catheter. It is further contemplated that the catheter may be provided with a side lumen to permit the withdrawal of spinal fluid for control of cerebrospinal fluid pressure. As the catheter is intended to extend the complete length of the spinal canal, the catheter will have a length of approximately 3 feet to provide ample catheter length for use during the procedure described below in greater detail. While specific parameters regarding the length and diameter of the catheter are presented herein in accordance with describing a preferred embodiment of the present invention, those skilled in the art will appreciate that these parameters may be varied to suit specific applications without departing from the spirit of the present invention.

With the catheter structure described above in mind, and in contrast to Khanna, the present cooling catheter 10 is well suited for percutaneous placement. As will be described below in greater detail, percutaneous placement of the present catheter 10 adds to the enhanced functionality of the present invention which results in a device specifically suited for cooling the spinal cord.

In addition, and further in contrast to Khanna, it has been found that it is desirable to provide a catheter without a heat exchanger. In particular, the entire catheter is positioned within the spinal canal and the entire catheter therefore cools the spinal cord. As such, the provision of a distal heat exchanger as disclosed by Khanna would be contrary to the intention of the present invention.

With regard to the cooling system 11 providing the cooling fluid to the catheter 10, a coolant fluid source 40 supplies coolant fluid to the catheter while maintaining the temperature of the coolant fluid at a predetermined temperature. For example, and in accordance with a preferred embodiment of the present invention, the coolant fluid is maintained at a temperature of −10° C. and

is generally composed of an ice and a supersaturated salt solution stored within an insulated container 42. With regard to the cooling fluid that has passed through the catheter, it is collected within an outlet collection tank 44. Tubing 32, 34 is provided for selective connection to the inlet channel 14, outlet channel 16, coolant fluid source 40 and outlet collection tank 44. The tubing 32, 34 is insulated to minimize thermal loss prior to passage of the coolant fluid within the catheter.

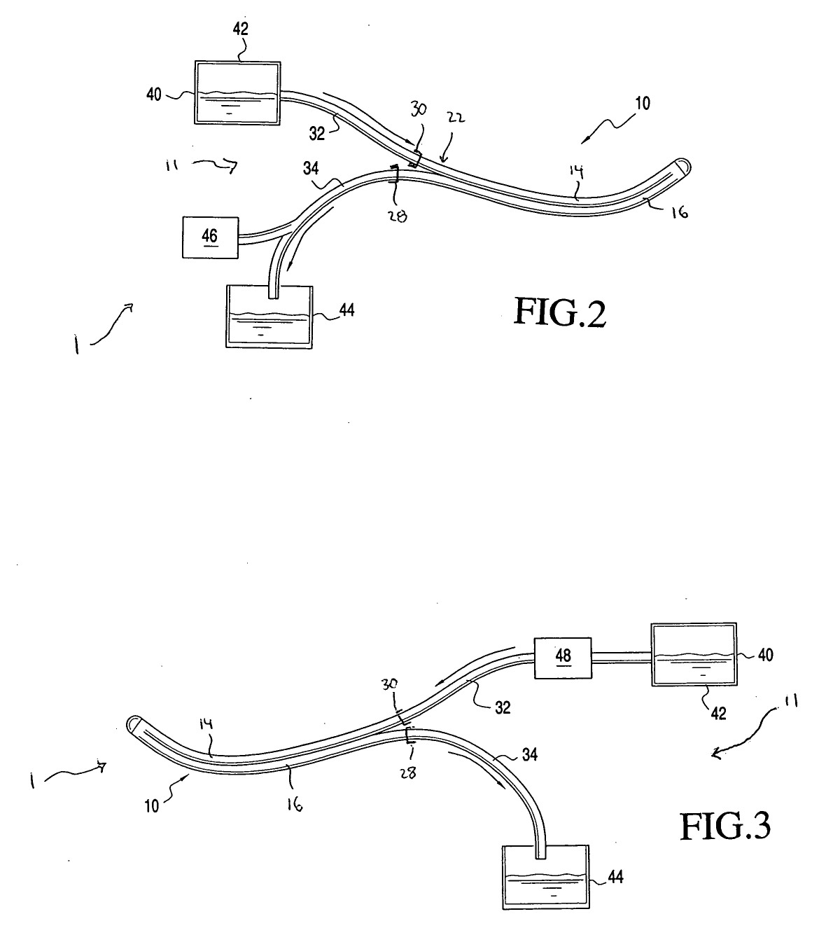

In accordance with preferred embodiments, two variations are contemplated for achieving fluid circulation. In accordance with a first embodiment, and with reference to FIG. 2, the coolant fluid will flow under a vacuum. In particular, the coolant fluid is drawn through the inlet and outlet channels 14, 16 via negative pressure bias. The vacuum 46 is applied to the outlet channel 16. The inlet tubing 32 (in the coolant fluid source 40) has a weighted filter element (not shown) to prevent flow blockages.

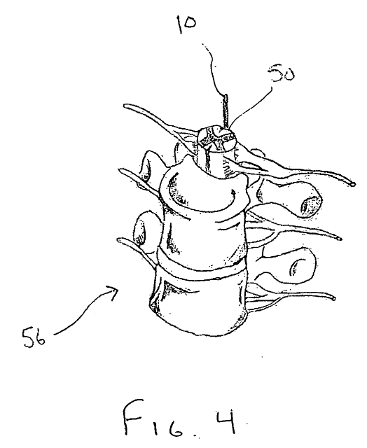

In accordance with an alternate embodiment, and with reference to FIG. 3, the coolant fluid flows under positive pressure from a pump 48. In particular, the coolant fluid is pushed through the inlet and outlet channels 14, 16 via positive pressure bias from a pump 48. As with the earlier embodiment, the inlet tubing 32 (in the coolant fluid source 40) has a weighted filter element (not shown) to prevent flow blockages. The pump 48 may be inside or outside of the coolant fluid source depending on specific requirements.

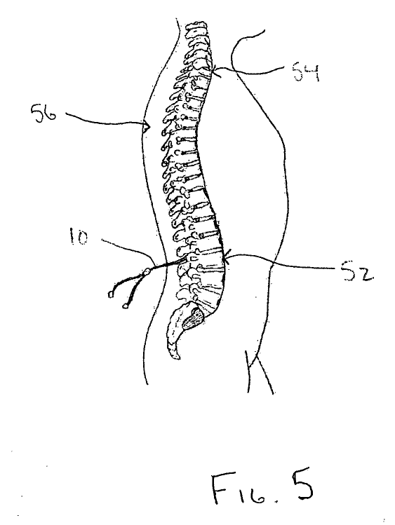

As discussed above, the present intra-thecal catheter system of the present invention is particularly adapted for application in therapy for descending thoracic aortic aneurysm surgery. In particular, and with reference to FIGS. 4, 5 and 6, the procedure is achieved by first anesthetizing and intubating the patient. The systemic temperature monitors (all conventional) are then positioned. In accordance with a preferred embodiment of the present invention an esophageal, nasopharyngeal and Foley monitor are employed, although other monitors may be used without departing from the spirit of the present invention.

The cooling catheter 10 of the present invention is then positioned within the spinal canal 50. In accordance with a preferred embodiment, the catheter 10 is placed so as to lie inside the intra-thecal space, from the lumbar site 52 of placement to a high thoracic level 54. Insertion is achieved percutaneously in much the same manner that a spinal catheter is traditionally inserted within the spinal canal. The catheter 10 is positioned within the spinal canal 50 to extend the entire length of the spine 56 and is maintained within the patient for 1 to 3 days as required, as is currently practiced with the non-cooling drainage catheters in widespread clinical use. During this time, the cooling system maintains a supply of cooling fluid to the catheter 10. In general, the cooler the spinal cord is maintained the better will be the protective results.

In accordance with a preferred embodiment, the spinal cord is cooled to a temperature as low as conceivably possible. While test results have shown the possibility of cooling the spinal cord to a temperature of approximately 28° C., it is known that exponential benefits are achieved as the spinal cord temperature is reduced. In fact, it is known that the desired fall in metabolic rate improves 50% for every 6° C. one is able to reduce the temperature of the spinal cord.

The benefits of cord hypothermia can also be expected to accrue to individuals with traumatic injury to the spine and spinal cord. Thus, the cooling catheter described in the present application may find additional usefulness, not only in patients undergoing surgery of the thoracic aorta, but also in non-surgical patients suffering injury to the spinal cord. Cooling of the intra-thecal space as described above will further provide benefits by similarly cooling the brain. In particular, by cooling the spinal canal, cerebrospinal fluid is cooled which in turn acts to cool the brain. This opens use of the present invention to patients with stroke affecting the brain or to those with mechanical trauma to the brain.

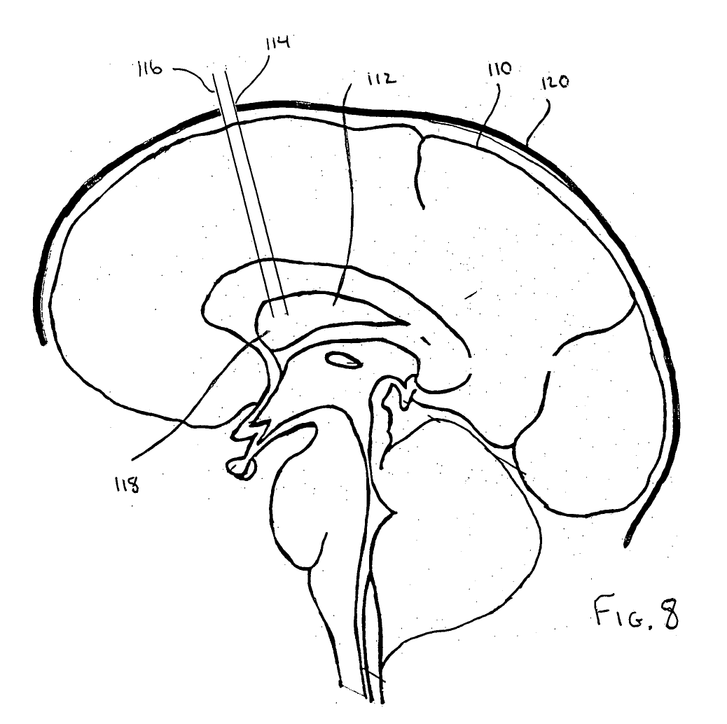

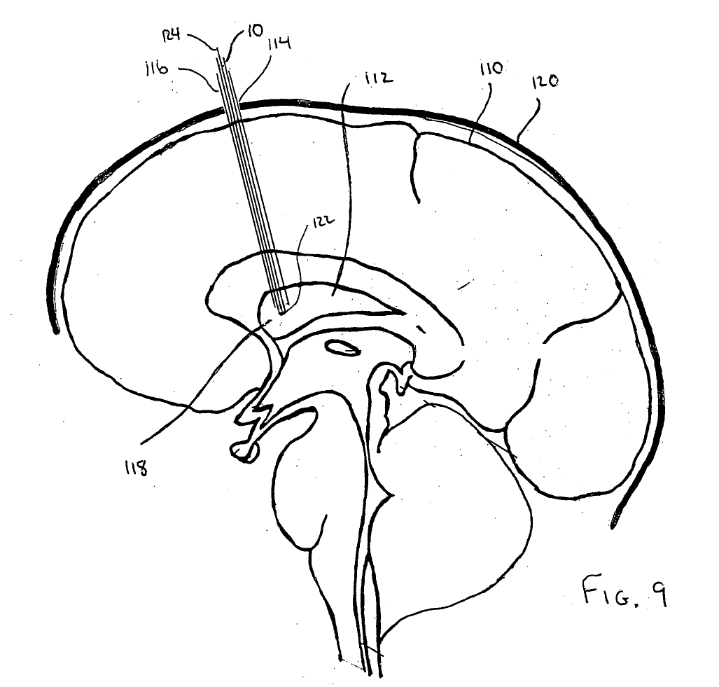

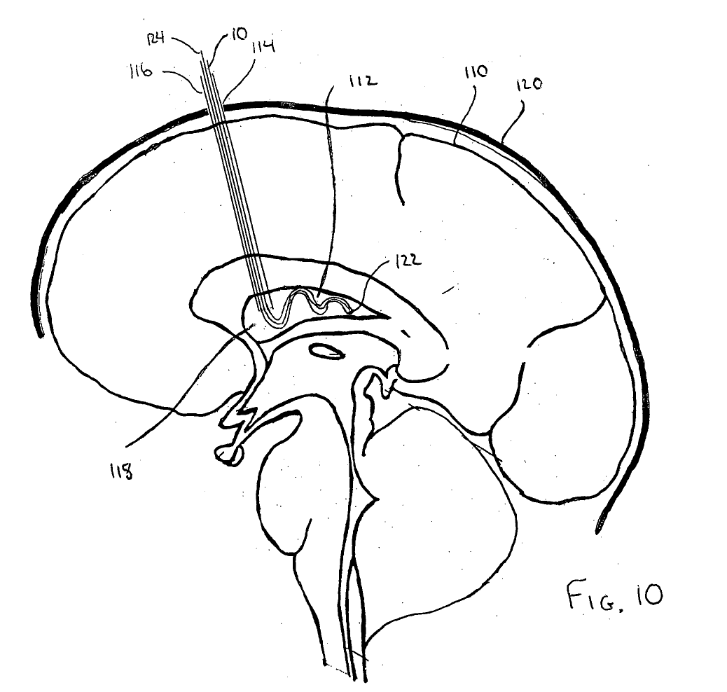

Referring to FIGS. 7 to 10, it is further contemplated the present catheter 10 may be used to provide hypothermic brain protection. Such brain protection would be provided in situations of cerebrovascular accident (for example, stroke) and traumatic brain injuries. In such situations, it is a standard neurosurgical practice to access one lateral ventricle 112 of the brain 110 via a burr hole 114 and a directed needle 116 puncture. As those skilled in the art will certainly appreciate, the lateral ventricles 112 form a portion of the ventricular system of the brain 110 and contain a reservoir of cerebral spinal fluid. In particular, the lateral ventricles 112 connect to the central third ventricle through the interventricular foramina of Monro.

In accordance with a preferred embodiment of the present invention, and with reference to FIGS. 7 to 10, a burr hole 114 is first formed in the skull 120 in accordance with traditional medical procedures those skilled in the art will certainly appreciate. The lateral ventricle 112 is then accessed via the burr hole 114 and the directed needle 116 puncture, the present catheter 10 is inserted through the needle 116 and into the ventricular cavity 118. For use in accordance with this procedure, the catheter 10 is shaped and dimensioned such that it will coil when positioned within the ventricular cavity 118. Once the catheter 10 is properly positioned, cooling fluid is recirculated through the lumens of the catheter 10 as described above in accordance with spinal cord applications. In general, and as discussed above with the spinal cord applications, the ventricular cavity 118 is preferably cooled to a temperature of approximately 28° C. and maintained at this temperature for 1 to 3 days as required.

In this way, the present procedure “spot cools” within the lateral ventricle 112 where cerebral spinal fluid is first encountered after passing through the grey and white matter of the brain. As such, cerebral spinal fluid is cooled, thus cooling the brain as well. By cooling the brain, protection is provided since it is well known that hypothermia of even modest proportions (even fractions of a degree) is highly brain protective. Through the utilization of this technique, a brain may be protected in cases of stroke or trauma.

Improved functionality of the catheter 10 in the performance of this procedure may be achieved by incorporating a monitor, for example, a fiber optic element 122, for measuring intracranial pressure and a ventricular drain 124 to release intracranial pressure when necessary by draining cerebral spinal fluid.

While the preferred embodiments have been shown and described, it will be understood that there is no intent to limit the invention by such disclosure, but rather, is intended to cover all modifications and alternate constructions falling within the spirit and scope of the invention.

1. A method for cooling of the brain, comprising the following steps:
positioning a cooling catheter within a ventricular cavity of the brain, the catheter including an inlet channel and outlet channel providing for the closed flow of cooling fluid into and out of the catheter;
cooling the catheter and ventricular cavity through the closed flow of cooling fluid through the catheter.

2. The method according to claim 1, wherein the ventricular cavity is that of a lateral ventricle of the brain.

3. The method according to claim 1, wherein the step of positioning includes accessing the ventricular cavity via a burr hole.

4. The method according to claim 1, wherein the step of cooling includes cooling the ventricular cavity to a temperature of at least 28° C.

5. The method according to claim 1, wherein the step of cooling includes cooling for approximately 1 day to 3 days.

6. The method according to claim 1, wherein the catheter includes a monitor measuring intracranial pressure.

7. The method according to claim 1, wherein the catheter includes a ventricular drain.

8. A method for cooling of the brain, comprising the following steps:
positioning a cooling catheter within a spinal canal, the catheter including an inlet channel and outlet channel providing for the closed flow of cooling fluid into and out of the catheter;
cooling the catheter and brain through the closed flow of cooling fluid through the catheter.

9. The method according to claim 8, wherein the step of positioning includes percutaneously inserting the catheter within the spinal canal.

10. The method according to claim 9, wherein the step of positioning includes placing the catheter along substantially the entire length of the spinal canal.

11. The method according to claim 9, wherein the step of cooling includes cooling the spinal cord to a temperature of at least 28° C.

12. The method according to claim 8, wherein the step of positioning includes placing the catheter along substantially the entire length of the spinal canal.

13. The method according to claim 8, wherein the step of positioning includes placing the catheter within the intra-thecal space.

14. The method according to claim 8, wherein the step of cooling includes cooling the spinal cord to a temperature of at least 28° C.

15. The method according to claim 8, wherein the step of cooling includes cooling for approximately 1 day to 3 days.
